(19) United States
(12) Patent Application Publication
(10) Pub. No.: US 2025/0134653 A1
(43) Pub. Date: May 1, 2025

(54) ENDOVASCULAR AORTIC ROOT REPAIR DEVICE

(71) Applicants: University of Maryland, Baltimore, Baltimore, MD (US); University of Maryland Medical System Corporation, Linthicum, MD (US)

(72) Inventors: Mehrdad GHOREISHI, Bethesda, MD (US); Bradley TAYLOR, Monkton, MD (US); Shahab TOURSAVADKOHI, Mt. Airy, MD (US); Aakash Yogesh SHAH, Laurel, MD (US)

(21) Appl. No.: 18/915,260

(22) Filed: Oct. 14, 2024

Related U.S. Application Data
(60) Provisional application No. 63/543,960, filed on Oct. 13, 2023.

(51) Int. Cl. A61F 2/24

(52) U.S. Cl. CPC A61F 2/2418; A61F 2/243

(57) ABSTRACT

An apparatus for endovascular repair of aorta root includes a self-expanding valved conduit and a self-expanding non-valved conduit. The valved conduit is configured for endovascular placement within an aortic valve of a subject. The valved conduit includes a valve between a distal end and a proximal end and includes a fenestration disposed between the valve and the proximal end of the valved conduit. The fenestration is configured for allowing fluid flow from inside the valved conduit to a coronary artery of the subject. The non-valved conduit is configured for endovascular placement within an aortic root of the subject. The proximal end of the valved conduit is fixed inside a distal end of the non-valved conduit and the distal end of the valved conduit extends outside the distal end of the non-valved conduit. The non-valved conduit also includes a fenestration in fluid communication with the fenestration in the valved conduit.

### CROSS-REFERENCE TO RELATED APPLICATIONS

This application claims benefit of Provisional Appln. 63/543,960, filed Oct. 13, 2023, the entire contents of which are hereby incorporated by reference as if fully set forth herein, under 35 U.S.C. § 119(c).

### BACKGROUND

Recent advances in endovascular aortic interventions and transcatheter aortic valve replacement (TAVR) management have introduced several theoretical endovascular treatment options for aortic pathologies that extend into the aortic root [1-3]. The aortic root is the beginning of the aorta, the body's largest artery, where the aorta attaches to the heart. The aortic root contains the aortic valve and the origins of the coronary arteries, which supply blood to the heart, as shown in FIG. 1. The medical term endovascular, literally “inside the blood vessel,” denotes a minimally invasive procedure that involves inserting a catheter into a blood vessel to diagnose and treat vascular conditions. Transcatheter means through a catheter.

Aortic root pathology is traditionally managed with the Bentall procedure where the aortic root is excised and replaced with a valved conduit with coronary reimplantation. That is, the Bentall procedure is an open-heart procedure through a large incision in the chest that replaces the aortic valve and part of the aorta with a synthetic graft, and re-implants the coronary arteries into the graft. For patients who are extremely high-risk for open Bentall procedure, especially in the setting of an acute type A aortic dissection (ATAAD), a less-invasive alternative may be beneficial. ATAAD is a life-threatening condition that occurs when the innermost layer of the ascending aorta tears, creating a false and true lumen. It's a surgical emergency that requires immediate intervention to prevent potentially fatal complications. Endovascular treatment of pathologies involving the aortic root for high-risk patients with ATAAD has not been described before. Thus, there remains a need to resolve aortic root repair.

Thoracic Endovascular Aortic Repair (TEVAR) is a minimally invasive surgical procedure that treats thoracic aortic aneurysms (excessive localized enlargement of an artery caused by a weakening of the artery wall) and dissections (tears). It often involves a covered stent (conduit made of a metal mesh tube and fluid-tight fabric) that serves as an arterial wall graft. An Endo-Bentall procedure uses a custom-made device to treat acute type A aortic dissection (ATAAD). The device integrates a self-expanding transcatheter aortic valve (TAVR) with a tapered endovascular stent graft (TEVAR) and wire-reinforced fenestrations for coronary artery stenting. The custom device is delivered in an endovascular procedure.

Only one single-stage composite Endo-Bentall procedure has been published to date (1). The first attempt was reported by Gaia and colleagues in 2020 (1) in a 64-year-old woman who presented with a bleeding suprasternal fistula (abnormal artery connection above the sternum) from a suture line pseudoaneurysm following conventional aortic valve replacement. A patient-specific balloon expandable composite graft was custom manufactured based on the patient's anatomy. The device had a stent graft measuring 34 millimeters (mm, 1 mm=10⁻³ meters)×40 mm with 2 branch grafts extending 2 cm from each coronary ostia (opening) for coronary stenting, and a balloon-expandable aortic valve. The device was delivered transapically (via a small opening in the chest) through a 30 Fr introducer. After device deployment, the procedure required the assistance of extracorporeal membrane oxygenation during coronary stenting. Extracorporcal membrane oxygenation (ECMO) is a machine that temporarily replaces the function of the heart and lungs. The procedure was successful, and the patient was alive without significant morbidity at 9 months following the operation.

The durability of covered stents in coronary arteries has only been shown in cases of iatrogenic (unintentional, induced by a physician) coronary rupture (6). Further studies are required to investigate the longevity of coronary stents when implanted through the manufactured openings (fenestrations) of a TEVAR graft stent and the metal struts of a TAVR device.

A simultaneous deployment of an ascending physician-modified TEVAR stent graft and TAVR was described by Gandet and colleagues (2) in an 82-year-old woman due to an expanding ascending aortic aneurysm extending into her aortic arch. The patient had prior TEVAR with carotid-subclavian bypass for a ruptured thoracic aortic aneurysm and subsequently developed a type IA endoleak (that occurs when blood continues to flow into an aneurysm sac after a graft) causing further expansion of her arch to 10 centimeters (cm, 1 cm=10⁻² meters). She was not a candidate for additional endovascular aortic arch intervention given a 5.5 cm ascending aortic aneurysm and lack of a proximal landing zone. Gandet and colleagues (2) performed a simultaneous physician-modified graft deployment from the femoral artery into the left ventricular outflow tract (LVOT) with a Sapien 3 TAVR valve (Edwards Lifesciences, Irvine, CA) deployment though the left ventricular apex via left thoracotomy (incision through chest between ribs) on ECMO support. The physician-modified graft was a custom-made graft stent with 2 antegrade side arms for the patient's innominate and left carotid arteries and had 2 fenestrations for perfusion into the left and right coronary arteries. Thus, the TAVR was inserted through the thoracotomy while at the same time the TAVER was inserted via catheter through the femoral artery. The patient suffered a left-sided stroke resulting in right-sided hemiparesis and dysphasia, and she was transferred to a neurological rehabilitation center on postoperative day 10.

A multi-stage Endo-Bentall was reported by Vallée and colleagues (3) performed in a 69-yar-old woman for an enlarging aortic aneurysm that involved the aortic root. The patient had a history of aortic valve replacement with wrapping of the ascending aorta 12 years prior. She was deemed a non-surgical candidate given impaired heart function and additional comorbidities at the time of presentation. Vallée and colleagues (3) initially tested the procedure on a 3D model and staged the Endo-Bentall procedure into distinct steps. They first deployed a tapered branched thoracic arch endo graft into the ascending aorta that extended across the prior bioprosthetic valve into the left ventricle. This was followed by a valve-in-valve with a 20 mm Sapien 3 TAVR valve (Edwards Lifesciences, Irvine, CA) that established the proximal seal for the branched covered stent. The final stage was cannulation and stenting of the coronary arteries through the branched thoracic artery stent graft. The patient unfortunately died post operative day 2 due to multisystem organ failure. Leshnower et al. (5) in a multi-staged device successfully sealed off the fistula from the noncoronary sinus of Valsalva to the left atrium in a 71-year-old man who presented with heart failure. They first stage deployed a modified TEVAR graft across the LVOT. In the next stage, the coronary arteries were accessed. A balloon expandable TAVR valve was deployed inside the previously placed TEVAR graft. The patient required cardiopulmonary bypass during the procedure because of severe aortic insufficiency (AI) created by deployment of the TEVAR graft across the LVOT.

Typical devices are often used with ECMO. For example, others have described use of a balloon-expandable TAVR deployed in a modular fashion after a fenestrated TEVAR within the LVOT (3,5).

### SUMMARY

The use of cardiopulmonary bypass via ECMO has been necessary to deal with the resultant severe aortic insufficiency (AI) and to support the perfusion of blood into the coronary arteries. One of the main challenges faced with use of a self-expanding TAVR device, was accessing the coronaries through the small strut of the valve frame. Described here, according to several embodiments, is an Endo-Bentall device and method for patients with ATAAD and other defects who are deemed too high-risk for conventional open-heart surgery. The device and method are customizable to improve safety and effectiveness of grafts, including physician-modified composite graft, from typical aortic stent grafts and valves such as TAVR devices.

Thus, in general, techniques are provided for endovascular aortic root repair. In a first set of embodiments, an apparatus includes a self-expanding valved conduit configured for endovascular placement within an aortic valve of a subject. The valved conduit includes a valve between a distal end of the valved conduit and a proximal end of the valved conduit. The valved conduit also includes one or more fenestrations disposed between the valve and the proximal end of the valved conduit. These one or more fenestrations are configured for allowing fluid flow from inside the valved conduit to a coronary artery of the subject. The apparatus also includes a self-expanding non-valved conduit configured for endovascular placement within an aortic root of the subject. The proximal end of the valved conduit is fixed inside a distal end of the non-valved conduit, and the distal end of the valved conduit extends outside the distal end of the non-valved conduit. One or more fenestrations are disposed in the non-valved conduit, each in fluid communication with a corresponding one of the fenestrations in the valved conduit. In some of these embodiments the valved conduit and non-valved conduit are each an off-the-shelf unit available in the marketplace.

In some embodiments of the first set, the fenestration disposed in the non-valved conduit is wire-reinforced.

In some embodiments of the first set, the distal end of the non-valved conduit is tapered smaller than a proximal end of the non-valved conduit.

In some embodiments of the first set, the proximal end of the valved conduit is fixed inside the distal end of the non-valved conduit by a suture disposed between the valve and the proximal end of the valved conduit, which suture attaches the valved conduit to the non-valved conduit.

In some embodiments of the first set, the apparatus is configured to be compressed inside an endovascular delivery subsystem.

In another set of embodiments, a system includes the apparatus and the endovascular delivery subsystem, wherein the apparatus is compressed inside the endovascular delivery subsystem.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTON OF THE DRAWINGS

Embodiments are illustrated by way of example, and not by way of limitation, in the figures of the accompanying drawings in which like reference numerals refer to similar elements and in which:

FIG. 1 is a block diagram of a human heart showing anatomical features for which a device is configured, according to an embodiment;

FIG. 2 is a block diagram that illustrates plan and side views of an example of a self-expanding valve-enclosing conduit, according to an embodiment;

FIG. 3 is a block diagram that illustrates an example of a single-stage self-expanding valve-enclosing graft, according to an embodiment;

FIG. 4A through FIG. 4D are block diagrams that illustrate an example of a method to deploy a single-stage self-expanding valve-enclosing graft, according to an embodiment;

FIG. 4E and FIG. 4F are block diagrams that illustrate an example of a method to deploy a later stage endovascular stent through the deployed graft of FIG. 4D, according to an embodiment;

FIG. 5 is a flow chart that illustrates an example of a method to make and use the graft of FIG. 3, according to an embodiment;

FIG. 6A and FIG. 6B are images that illustrate examples of measurements of anatomical features of a subject in which to deploy the graft of FIG. 3, according to an embodiment;

FIG. 7A through FIG. 7D are images that illustrate conditions of two subjects prior to deployment of respective embodiments of the graft of FIG. 3, according to two embodiments;

FIG. 8A through FIG. 8H are images that illustrate 3-dimensional digital and physical models of the anatomy of two subjects used to customize embodiments of the graft of FIG. 3, according to two embodiments;

FIG. 9A through FIG. 9G are photographs that illustrate an example method for fabricating an embodiment of the graft of FIG. 3, according to an embodiment;

FIG. 10A through FIG. 10E are photographs that illustrate an example method for collapsing the embodiment of the graft of FIG. 3 into an endovascular delivery system, according to an embodiment; and

FIG. 11A through FIG. 11E are photographs that illustrate proper positioning of an embodiment of the graft of FIG. 3 and adequate sealing of a false lumen after subsequent deployment of covered stents into coronary arteries, according to an embodiment.

### DETAILED DESCRIPTION

A method and apparatus are described for repair of damage to an aortic root. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of melding a TAVR valve device with a TEVAR covered stent graft in human subjects. However, the invention is not limited to this context. In other embodiments other self-expanding valve-including conduits are melded with other self-expanding non-valve conduits to form a single-stage endovascular aortic valve and root repair device in human and non-human subjects.

### 1. OVERVIEW

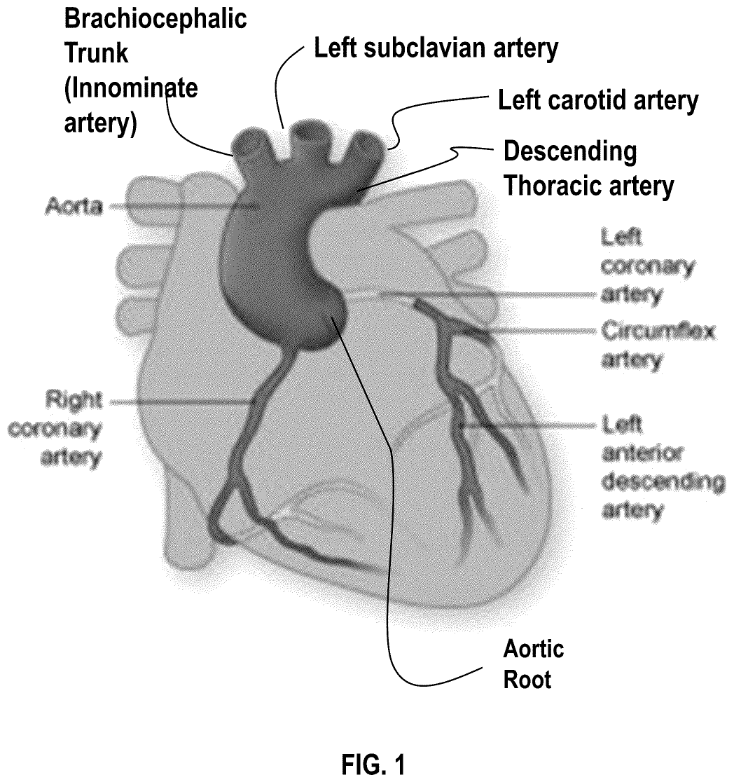

FIG. 1 is a block diagram of a human heart showing anatomical features for which a device is configured, according to an embodiment. The aortic root includes the aortic valve allowing flow from the left ventricle of the heart into the aorta and preventing backflow into the left ventricle. The aortic root also includes connections to the left and right coronary arteries. In a blood flow direction beyond the root is the ascending portion of the aorta which is followed by the aortic arch followed by the descending thoracic artery. The aortic arch has a connection to the Brachiocephalic trunk, also called the Innominate artery, followed by a connection to the left subclavian artery followed by a connection to the left carotid artery. The Brachiocephalic trunk supplies blood to the head, neck and upper extremities on the right side of the subject through subsequent branches called right subclavian artery that supplies blood to the to the right arm, and the right carotid artery that supplies blood to the right neck and head. The left subclavian artery supplies blood to the to the left arm. The left carotid artery supplies blood to the left neck and head.

### 1.1 System Structures

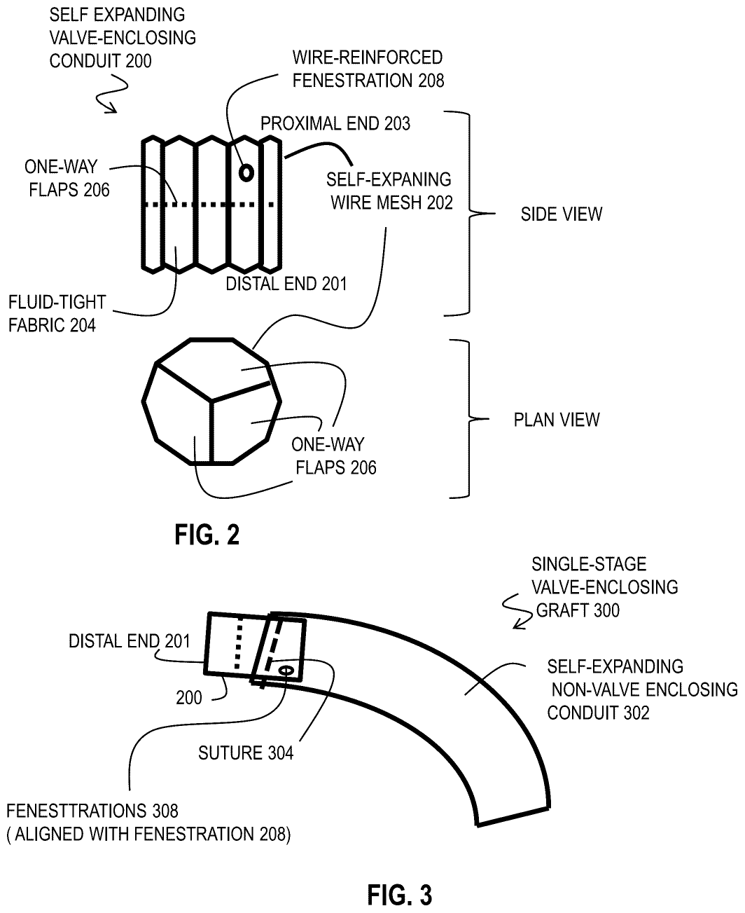

FIG. 2 is a block diagram that illustrates plan and side views of an example of a self-expanding valve-enclosing conduit 200, according to an embodiment. The valve enclosing conduit, also called a valved conduit herein for simplicity, such as a TAVR device, includes a self-expanding wire mesh 202 covered with a fluid tight fabric 204. A distal end 201 of conduit 200 is configured to extend through the aortic valve of a subject and into the left ventricle of the subject. A proximal end 203 of conduit 200 is configured to extend into the aortic root that is beyond the aortic valve of the subject in the direction of desired blood flow. Between the distal and proximal ends, transverse to an axis of the conduit is disposed a plurality of one-way flaps 206 (also call leaflets), typically three flaps, that are configured to prevent backflow into the left ventricle. A gap cut in the fabric 204 between wires of the wire mesh 202 is called a fenestration. Such a fenestration is configured for allowing fluid flow from inside the valved conduit to a coronary artery of the subject. According to some embodiments the valved conduit 200 includes one or more wire-reinforced fenestrations 208.

FIG. 3 is a block diagram that illustrates an example of a single-stage self-expanding valve-enclosing graft device 300, according to an embodiment. The device 300 includes the valved conduit 200 affixed to a self-expanding conduit 302, such as a TEVAR stent graft, that does not include a valve. Such a conduit is called herein a non-valved conduit for simplicity. The proximal end of the valved conduit is fixed inside a distal end of the non-valved conduit and the distal end of the valved conduit extends outside the distal end of the non-valved conduit. In this embodiment, a wire-reinforced fenestration 308 is disposed in the non-valved conduit 302 in fluid communication with the fenestration 208 in the valved conduit 200. In some embodiments the fenestrations 208 and 308 are formed at the same time. In some embodiments the fenestrations 208 and 308 are configured such that a coronary stent can be deployed through them both.

Any means may be used to affix the valved conduit to the non-valved conduit such as gluing or soldering the wire meshes. In an example embodiment, the valved conduit 200 is affixed to the non-valved conduit with a suture 304 passing alternately through each conduit mesh circumferentially around each conduit.

### 1.2 System Methods

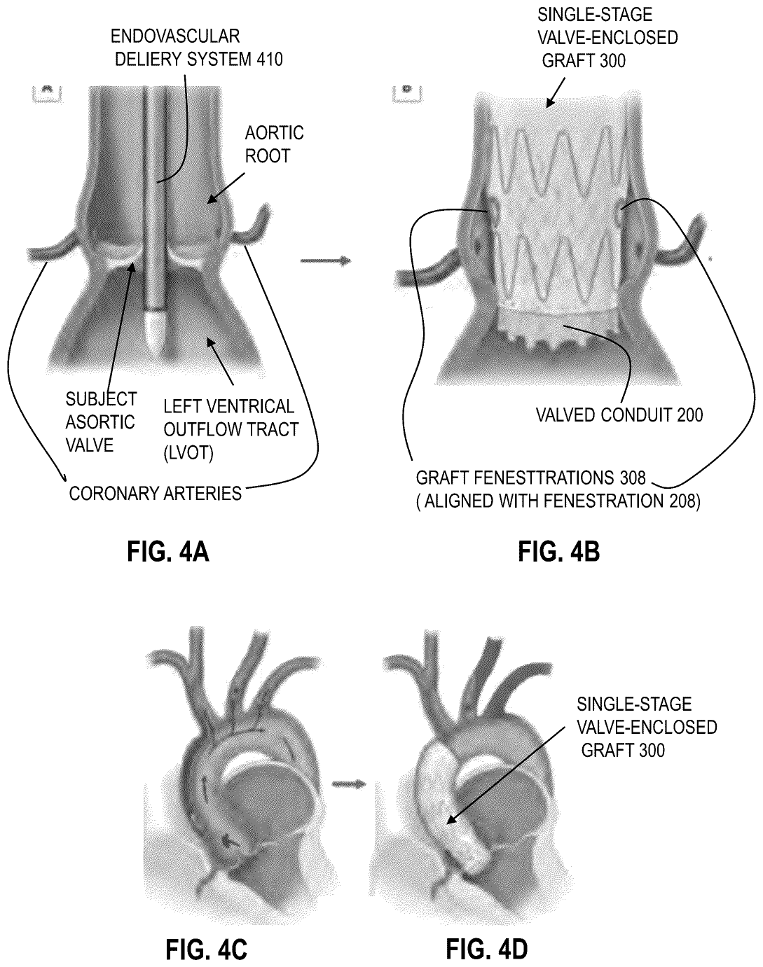

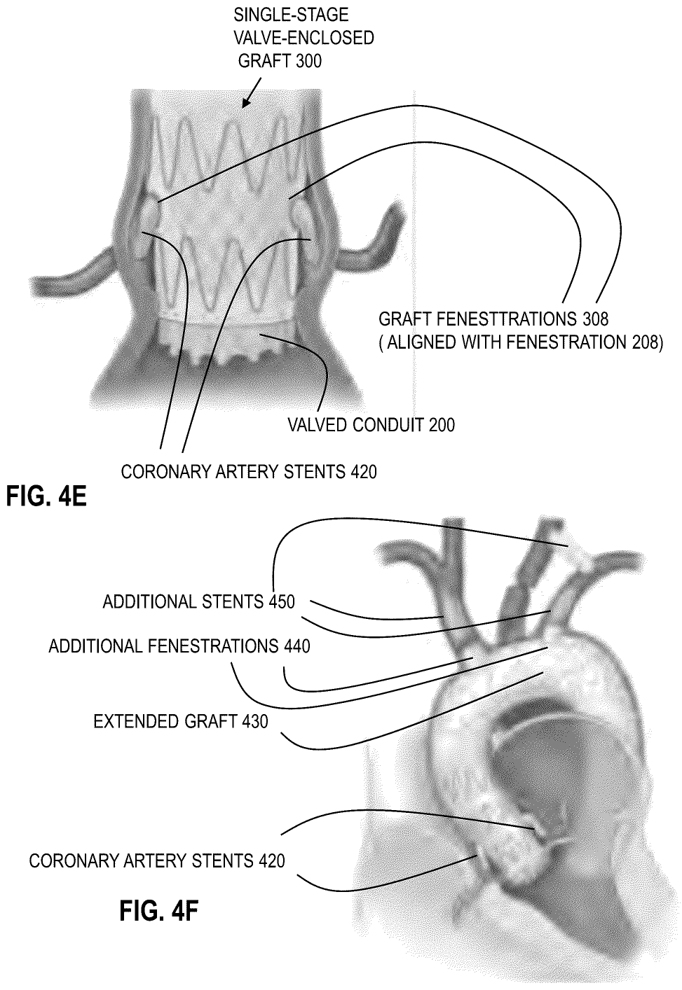

FIG. 4A through FIG. 4D are block diagrams that illustrate an example of a method to deploy a single-stage self-expanding valve-enclosing graft device 300, according to an embodiment. FIG. 4A, depicts the left ventricle outflow tract (LVOT) of a subject, the aortic valve of the subject, and the aortic root of the subject, along with right and left coronary arteries. Also depicted is an endovascular delivery system 410, such as a TEVAR delivery system. A nosecone tip of a catheter is fed through an artery until it penetrates the aortic valve of the subject into the LVOT of the subject. FIG. 4B depicts the single-stage valve-enclosed graft 300 after removal of the delivery system and self-expansion. As can be seen, the distal end of the valved conduit 200 extends into the LVOT, at a position to displace the subject's aortic valve. The graft device 300 fenestrations 308 aligned with the valved conduit 200 fenestrations 208 are in fluid communication with the coronary arteries. FIG. 4C depicts the aorta before the delivery of the graft device 300 and FIG. 4D illustrates the single-stage graft device 300 in place.

FIG. 4E and FIG. 4F are block diagrams that illustrate an example of a method to deploy endovascular stents through the deployed graft of FIG. 4D during a later stage, according to an embodiment. If it is determined, e.g., through symptoms or imaging observations, that perfusion into the coronary arteries is insufficient, e.g., due to a persistent endoleak, then stents can be deployed into the coronary arteries. As shown in FIG. 4E, coronary artery stents 420 are deployed into the coronary arteries through the fenestrations 208, 308 in the graft device 300. An advantage of wire-reinforced fenestrations 208 or 308 or both is that it becomes easier and more reliable to find and deploy the coronary stents 420 through the fenestrations 208 and 308 into the coronary arteries

If it is determined, e.g., through symptoms or imaging observations, that perfusion into any or all of the arteries connected to the aortic arch is insufficient, then, as depicted in FIG. 4F, one or more extended grafts 430 with additional fenestration 440 can be deployed in a later stage to be followed in a still later stage with additional stents 450 deployed through those fenestrations 440. In some embodiments, one or more fenestrations 440 are wire-reinforced fenestrations. In some embodiments, the non-valved conduit 320 of the single-stage valved graft 300 extends up to the full length of the extended graft 430 and includes one or more wire-reinforced fenestrations of the additional fenestration 440.

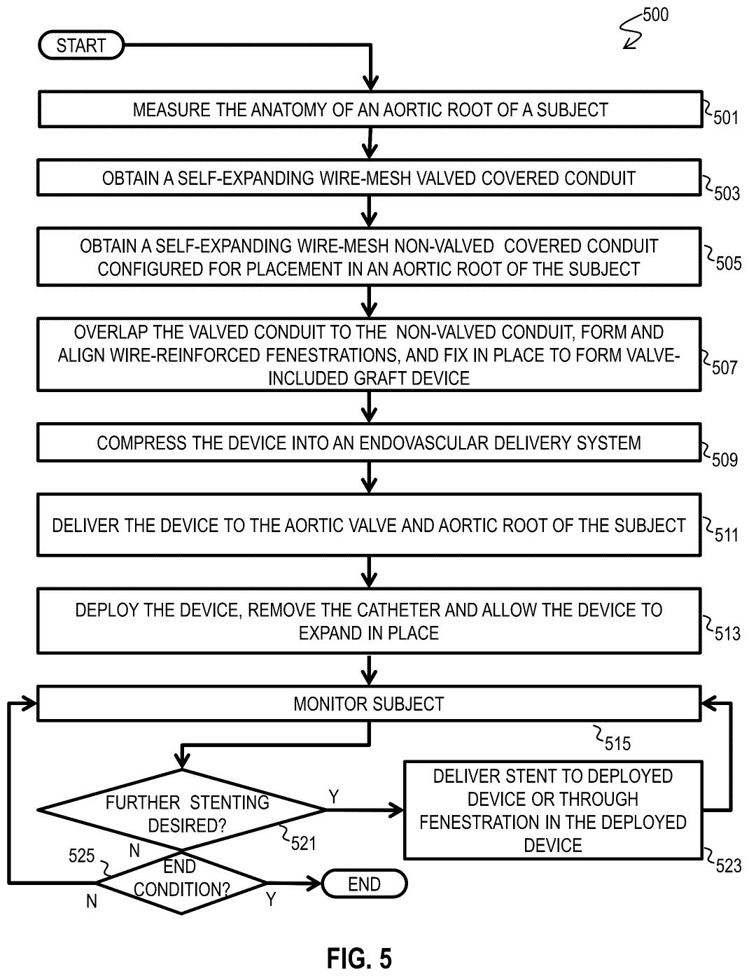

FIG. 5 is a flow chart that illustrates an example of a method 500 to make and use the graft device 300 of FIG. 3, according to an embodiment. Although the process is depicted in FIG. 5 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

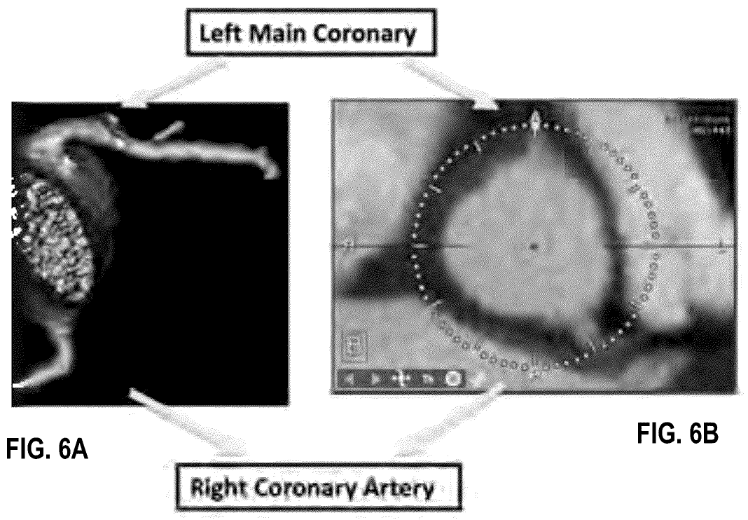

In step 501, the anatomy of a particular subject is determined to ascertain position of coronary arteries and extent of damage to aortic root. These anatomical features dictate positions for fenestrations 308 to supply the coronary arteries, length of the non-valved conduit 320, and need for any additional fenestrations in an extended graft 430 for one or more arteries branching from the aortic arch. Any methods may be used to determine the anatomy, including medical imaging with or without contrast, such as 2-dimensional X-rays, computed aided tomography (CAT) X-ray, magnetic resonance imaging (MRI), ultrasound (US), Doppler US, endoscopy, and catheterization. FIG. 6A and FIG. 6B are images that illustrate examples of measurements of anatomical features of a subject in which to deploy the graft of FIG. 3, according to an embodiment. These images of preoperative CT scans show the circumferential locations of left and right coronary arteries. This imagery enables coronary fenestrations 208 or 308 or both to be made in appropriate locations in graft device 300.

In some embodiments, step 501 includes printing a patient-specific 3-D model to simulate the endovascular repair, provide a platform for practice or provide the proof of concept of the technique, or some combination. For example, source images are obtained from the arterial phase acquisition of a routine Aortal 3-phase protocol (Somatom Force CT scanner, Siemens, Munich, Germany) axial reconstruction at 2 mm slice thickness at 1 mm interval. The aorta imaging sequence is imported into the Mimics Innovation Software Suite (Materialise, Leuven, Belgium) for segmentation (Supplement 1-A) and optimization for printing. The finished design is printed on a ProJet 660Pro color printer (3D Systems, Rock Hill, SC).

In step 503, a self-expanding wire-mesh valved covered conduit 200 is obtained, e.g., through purchase or fabrication. In step 505, a self-expanding wire-mesh non-valved covered conduit 302 is obtained, e.g., through purchase or fabrication. In some embodiments, the non-valved conduit 302 is modified as advantageous to fit the diameter, length, and longitudinal shape of the section of the aortic root and aortic arch to be repaired for the particular subject. In one embodiment, the valved conduit 200 of the device 300 is configured with a landing zone based on a patient's LVOT (8 mm to 1 cm below the aortic valve annulus). The size of the catheter valve determines the size of the end of the TEVAR graft (same size or approximately 1-2 mm oversized). A separate landing zone may be at the junction of the innominate artery and the ascending aorta if the ascending aorta is not aneurysmal. For patients with aneurysm involving the ascending aorta, an Endo-Arch procedure can be performed prior to Endo-Bentall in a staged fashion.

In step 507, the valved conduit 200 is overlapped with the non-valved conduit 302, to form or align, or both, all of one or more wire-reinforced fenestrations 208 and 308, and fix the two conduits 200 and 302 relative to each other to produce single-stage valve-included graft device 300. In some embodiments, step 505 includes forming the fenestration by adding a wire loop and puncturing the fluid-tight fabric inside the loop. Forming the fenestration also includes positioning the fenestrations based on the anatomy of the subject as determined in step 501 to supply access to the coronary arteries and any of the arteries in the aortic arch otherwise covered by the extended graft 430. According to several embodiments, the self-expandable TAVR valves can include a cuff (e.g., of pericardium) configured to secure the TEVAR graft to the TAVR frame without interfering with the valve leaflets (flaps), such as by extending proximal to the valve leaflets. Furthermore, delivering the TAVR valve coupled to the TEVAR can reduce the risk of significant arterial insufficiency (AI) during deployment, compared to typical devices (e.g., non-coupled TEVAR valve and TEVAR graft), thus reducing the need for a physician to require extracorporeal cardiopulmonary support.

In step 509, the single-stage valve-included graft device 300 is compressed and sheathed (or otherwise encapsulated) in an endovascular delivery system, such as TAVER or other catheter.

In step 511, a catheter of the endovascular delivery system is fed through an arm or leg artery, such as the femoral artery, and threaded into the LOVT to deliver the compressed single-stage valve-included graft device 300 to the aortic root. In some embodiments, step 511 includes positioning the graft to appropriately align the fenestrations with the left and right coronary ostia. Furthermore, one embodiment of the device can include confirming the desired position of the device and aligning the device (e.g., using fluoroscopy). For example, aligning can include the positioning device such that the device's fenestrations align with at least one of the left and right coronary ostia of a patient. The fenestrations are configured to allow for filling of the coronary sinuses perfusing the coronary arteries.

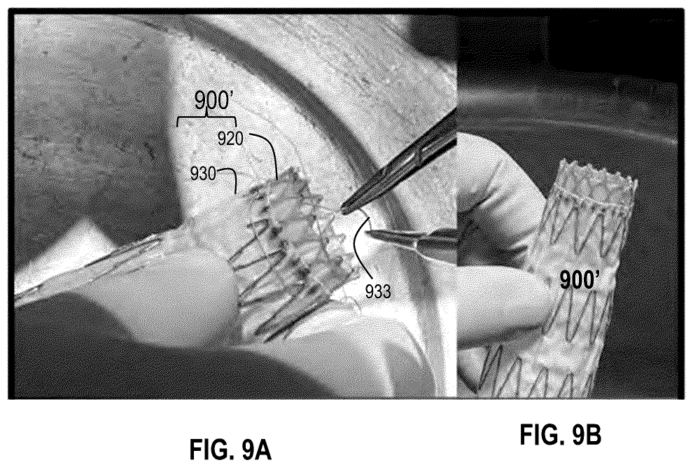

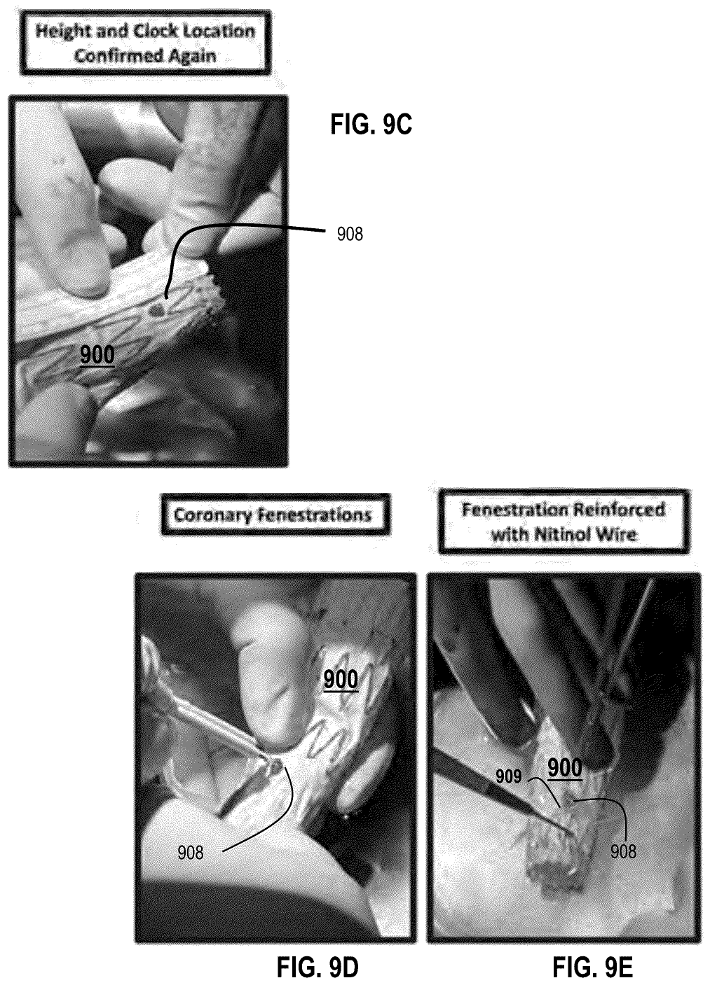

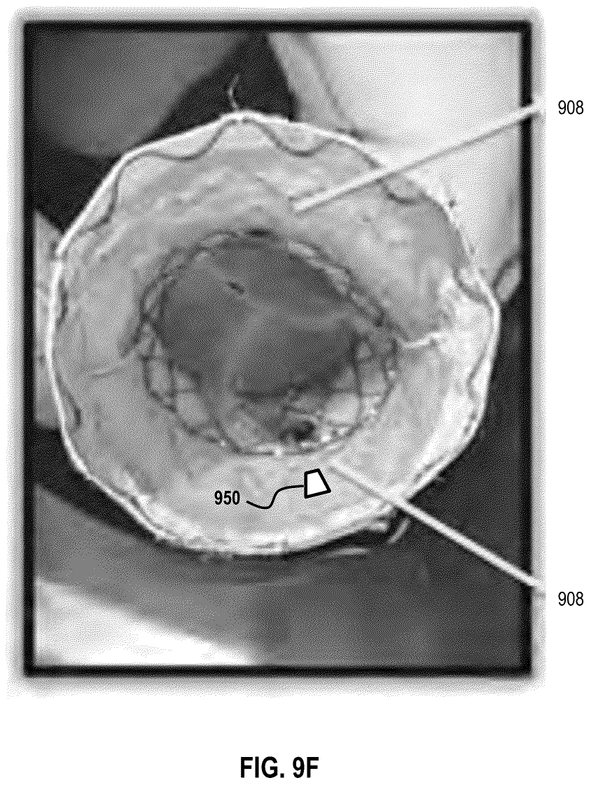

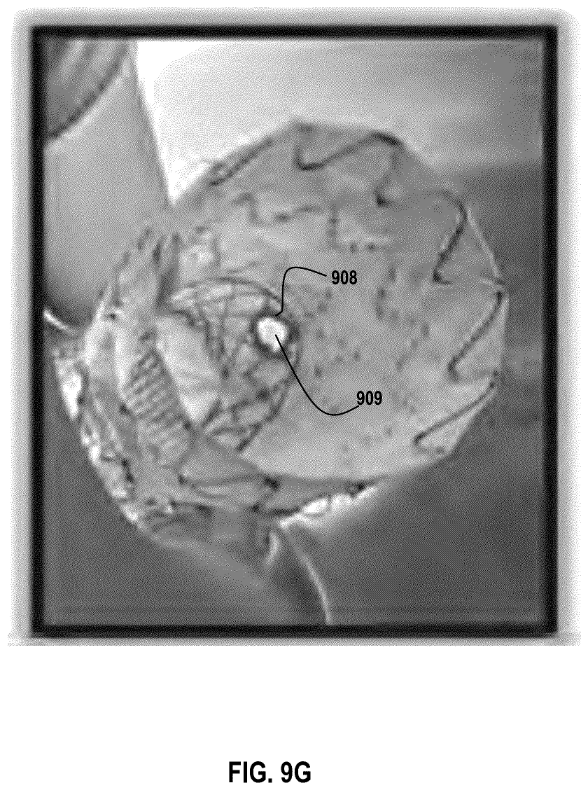

In another embodiment, the device 300 is configured to be visible using imaging techniques. For example, the device is configured to be visible when positioning the device in a patient. In one embodiment, the device includes at least one marker (e.g., marker 950 depicted in FIG. 9F, described below) that is visible to an imaging device. For example, the device can include a marker at a patient's left coronary bottom or hole. Coronary bottom is the same feature as the coronary hole or the coronary Ostia. Coronary cusp is a larger area of the aortic root where the coronary hole is located. (i.e. cusp). In another example, the device can include a marker at a patient's middle portion of a non-coronary path. In still another embodiment, the device can include a marker at a patient's right (or non-left or non-coronary) cusp. In still yet another embodiment, the device includes a combination of markers. Each marker can be a discrete point attached to the device, such as sown or adhered. In another example, a marker can be elongated, such as a linear marker that extends from a patient's right coronary to a base. The markers can be formed of typical materials visible under CT imaging, for example. Thus, the device is configured to be visible using imaging techniques to position and align the device 300, especially the coronary fenestrations 308, in a patient.

In step 513, the device 300 is deployed by releasing it from the catheter or other delivery system, removing the catheter or other delivery system, and allowing the device 300 to expand in place.

In step 515, the subject is monitored to determine that the desired outcome is achieved, e.g., that the aortic root is preventing endoleak and is supplying sufficient flow to the coronary and other branching arteries. Any method may be used to monitor the subject including ECG, medical imaging, endoscopy, further catheterization. In step 521, it is determined whether further stenting is desired or otherwise advantageous to repair any further conditions detected by the monitoring during step 515. If so, the process moves to step 523 to deliver any desired stent, such as a later stage extended graft 430 to the aorta itself, or a stent, such as coronary artery stents 420 to one or more coronary arteries, or one or more additional stent 450 to a target branching artery, through a wire-reinforced fenestration in the deployed device 300 or later stage graft 430. For example, if persistent endoleak is present, a physician can access the coronary ostia through the fenestrations for stenting. In another example, the method can include deploying the device when the patient is overdrive-paced. In at least one embodiment, no cardiopulmonary support is required. The method passes back to step 515 to monitor the subject.

In some embodiments, during step 523, the device 300 is configured to achieve coronary stenting through the graft fenestrations, rather than through parallel stents outside of a main device as is common in typical devices. Coronary stenting through the graft fenestrations simplifies the device, minimizes the coronary manipulation and wiring, and decreases the risk of stent embolization and kinking that can occur with coronary snorkeling, compared to typical devices. In another embodiment, the device is configured as a multi-staged modular devices such that a physician can perform coronary access and stenting before complete deployment of the TAVR valve. This can reduce the risk of coronary mal-perfusion if the device is mal-positioned. However, the (two) fenestrations of the graft configured to align with the coronary arteries can further reduce the risk of coronary mal-perfusion. For example, such risk increases when positioning and deploying the device causes significant malalignment to the pre-planned deployment plane such that neither fenestration aligns to the coronary cusps for coronary mal-perfusion to occur. Even if one fenestration aligns in front of one cusp, the device is configured to allow blood flow through the cusps and perfuse the other coronary. In another embodiment, placing constraining sutures in the TEVAR graft at the level of the coronary arteries can further increase the likelihood of adequate coronary perfusion after device deployment.

If it is determined, in step 521, that further stenting is not desired or not otherwise advantageous, then the method passes to step 525. In step 525, it is determined whether any stop condition is satisfied, e.g., the subject is released or expires. If not, the method passes back to step 515 to monitor the subject. If the end condition is satisfied, then the process ends.

Thus, one embodiment of the device 300 includes a single-staged endovascular valve-carrying conduit device for endovascular aortic root repair (Endo-Bentall) in Type A dissection. The device 300 can include multiple components, such as self-expanding transcatheter aortic valve (TAVR valve), a self-expanding tapered aortic endovascular stent graft, and at least one wire-reinforced fenestration for coronary artery stenting. Furthermore, the delivery and deployment of the device 300 can be used in a similar manner as other self-expanding TAVR valves to reduce the need for additional training. For example, the device 300 can be deployed through femoral artery access. In some embodiments, no cardiopulmonary bypass is required and thus no extra-corporal membrane oxygenation (ECMO) support is required. Thus, provided is an Endo-Bentall repair device with a single-staged valve-carrying conduit and method for high-risk patients with aortic root pathologies.

According to several embodiments, the Endo-Bentall device is a customizable device to improve safety and effectiveness of grafts, including physician-modified composite graft, from typical aortic stent grafts and TAVRs. One embodiment of the device includes a self-expanding transcatheter aortic valve (TAVR), a self-expanding endovascular stent graft (TEVAR), and at least one (e.g., two) wire-reinforced fenestrations for coronary artery stenting. The TAVR valve is incorporated into an appropriately sized TEVAR graft and sutured together circumferentially. The coronary fenestrations are made in the TEVAR graft. At least one embodiment of the Endo-Bentall device is configured to be re-sheathed within a TEVAR delivery system.

Such Endo-Bentall procedures are feasible options for patients with acute TAAD who are deemed too high risk for conventional open surgery. It is possible to construct a safer and more effective physician-modified composite graft from the available aortic stent grafts and valves that are currently on the market

### 2. EXAMPLE EMBODIMENTS

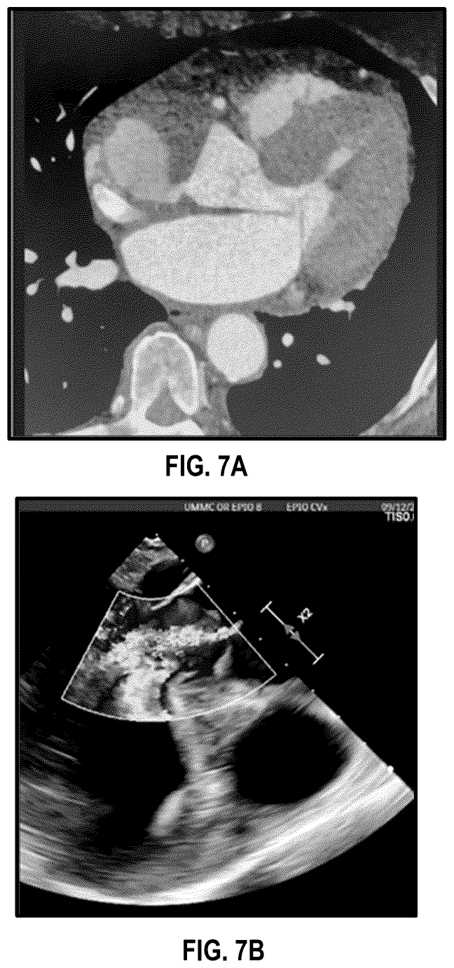

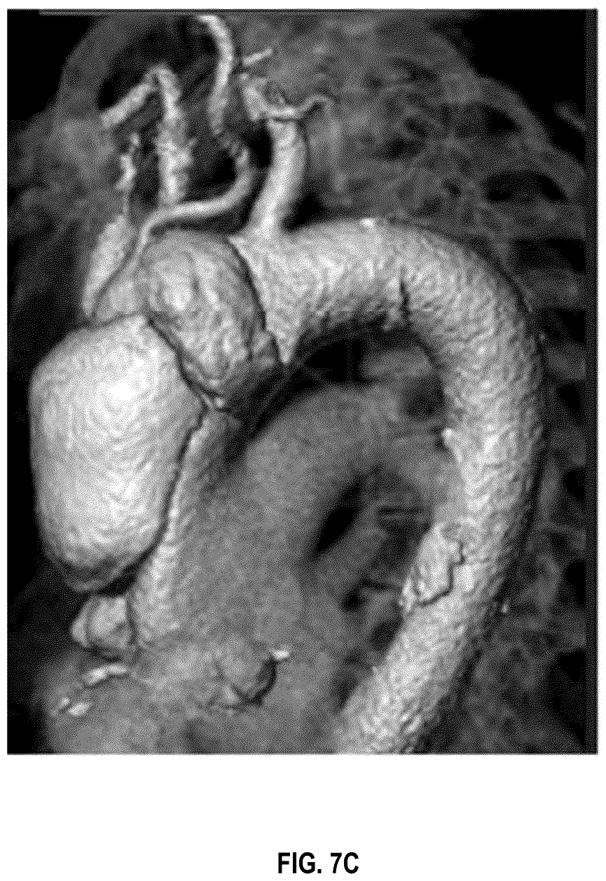

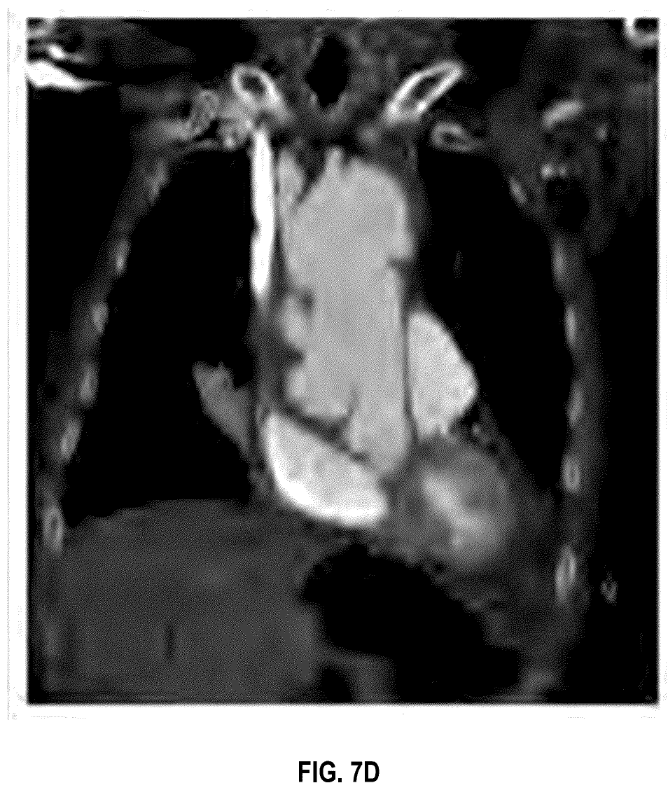

Two example devices 300 were constructed and demonstrated for two patients, respectively, using method 500 of FIG. 5. FIG. 7A through FIG. 7D are images that illustrate conditions of two subjects prior to deployment of respective embodiments of the graft of FIG. 3, according to two embodiments. Such measurements are collected during step 501.

Patient 1 was a 63-year-old woman presented to the emergency room with crushing chest pain and shortness of breath that started the night before the presentation. She had been diagnosed with stage IV endometrial cancer 3 years ago and was actively undergoing chemotherapy and immunotherapy. Chest computed tomography angiography. (CTA) showed ATAAD with a large fenestration between the true and false lumen at the level of the non-coronary cusp and intramural hematoma (IMH) extending into the level of the innominate artery. FIG. 7A depicts preoperative CTA showing acute TAAD with primary entry tear in the non-coronary cusp. FIG. 7B depicts preoperative transthoracic echocardiogram (TTE) showing severe aortic insufficiency (AI). The patient was deemed a non-surgical candidate given ongoing treatment for metastatic stage IV endometrial cancer. Ascending TEVAR intervention was not possible given the anatomical location of the entry tear and the extent of the IMH. She was deemed a candidate for Endo-Bentall intervention. A collaborative team of cardio-aortic surgeon, heart valve structure surgeon, interventional cardiologist, and vascular surgeon, was involved in the decision making, preoperative planning, and execution of the procedures. Endovascular management was discussed and approved by the patients, the hospital ethics committee, and the Institutional Review Boards (IRB).

Patient 2 was an 85-year-old female presented with acute chest pain. Chest CTA demonstrated a 6.3 cm ascending aortic aneurysm with multiple dissection flaps consistent with an acute on chronic type A aortic dissection originating at the sino-tubular junction extending into the aortic arch. There was aneurysmal dilation of the aortic root, ascending aorta and the aortic arch. FIG. 7C and FIG. 7D depict preoperative imagery and echocardiography, respectively, which showed ejection fraction of 55% and trace aortic insufficiency (AI). A multidisciplinary team convened and agreed that given the patient's age and poor functional status, she was at a prohibitive risk for open aortic root and total arch replacement. An alternative endovascular approach consisting of staged endovascular arch repair (Endo-Arch) followed by placement of an endovascular valved conduit with coronary stenting (Endo-Bentall) was determined to be feasible, similar to the deployed structures depicted in FIG. 4F. A collaborative team of cardio-aortic surgeon, heart valve structure surgeon, vascular surgeon, and interventional cardiologist was involved in the decision making, preoperative planning, and execution of the procedures. Endovascular management was discussed and approved by the patients, the hospital ethics committee, and the Institutional Review Boards (IRB).

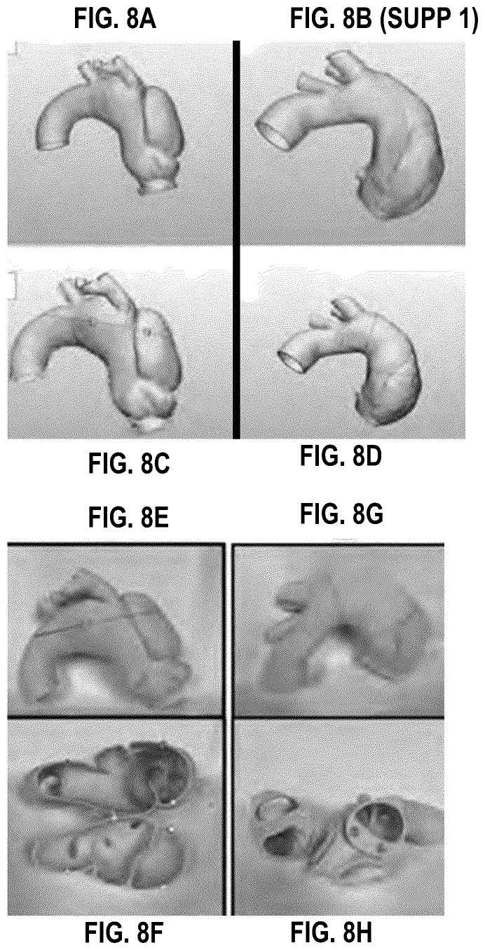

FIG. 8A through FIG. 8H are images that illustrate 3-dimensional digital and physical printed models of the anatomy of two subjects used to customize embodiments of the graft of FIG. 3, according to two embodiments. These representations are generated during step 501 described above. After obtaining 3-dimensional medical imaging and segmenting the imagery to identify the aortic root, ascending aorta and aortic arch, representative three-dimensional renderings of patient specific aortic arch were generated. FIG. 8A depicts digital representation for patient 1. FIG. 8B depicts digital representation for patient 2. After optimization for printing, representative three-dimensional rendering of patient specific aortic arch for patient 1 and patient 2 are depicted in FIG. 8C and FIG. 8D, respectively. A 3-dimensional printer model of patient specific aortic arch for patient 1 is depicted in assembled and split open views in FIG. 8E and 8F, respectively. A 3-dimensional printer model of patient specific aortic arch for patient 2 is depicted in assembled and split open views in FIG. 8G and 8H, respectively.

FIG. 9A through FIG. 9G are photographs that illustrate an example method for fabricating an embodiment 900 of the graft of FIG. 3, according to an embodiment. These illustrations correspond to steps 503 to 507 of method 500 depicted in FIG. 5.

According to some example embodiments, an Endo-Bentall device 900 (an embodiment of graft 300) includes a self-expanding TAVR valve as valved conduit 920, a self-expanding aortic endovascular stent graft (TEVAR) as non-valved conduit 930, obtained during steps 503 and 505, described above, respectively. The device 900 also includes at least one wire-reinforced fenestration 908, for coronary artery perfusion or stenting. For example, embodiment 900 of the device includes two wire-reinforced fenestrations 908.

The fenestrations 908 are configured to match positions of coronary arteries in a particular patient. For example, the fenestrations 908 can be sized and circumferentially located according to a computed tomography angiography (CTA)-TAVR protocol and measurement program (e.g., from TeraRecon, Durahm, NC, USA). Still further a CTA-TAVR and program can measure a patient's valve size, proximal seal zone in the left ventricular outflow tract (LVOT), and distal seal zone in the ascending aorta to configure the device 900 for a patient.

According to other embodiments, a TAVR valve (e.g., Evolut PRO; Medtronic) as valved conduit 920 is incorporated into an appropriately sized TEVAR stent graft (e.g., Zenith TX2, Cook) as non-valved conduit 930. In step 507, the TAVR valve and graft can be sutured together circumferentially, such as by using a 4-0 Ethibond (FIG. 9A) suture 933 to produce a combined but unpunctured conduit 900′ depicted in FIG. 9B. Fenestrations 908 are positioned as depicted in FIG. 9C, punctured as depicted in FIG. 9D, and wire reinforced with wire 909 as depicted in FIG. 9E to produce graft device 900. The fenestrations (left and right coronary) are sized based on CTA measurements and wire-reinforced with looped segments of nitinol guidewire 909. In one embodiment, the fenestrations allow for filling of the coronary sinuses perfusing the coronary arteries. FIG. 9F and FIG. 9G, show a view into the graft 900 from the proximal end of TEVAR conduit, showing fenestrations 908 in the overlap between the TAVR and the TEVAR conduits. FIG. 9G clearly shows the wire loop 909 reinforcing the fenestration 908. In some embodiments, the graft device 900 includes one or more markers 950 that can be sensed during delivery to ensure the graft 900 is rotated such that the fenestration 908 are close to, and presumed in fluid communication with, the coronary artery ostia (openings).

In step 509 of the method 500 described above, the Endo-Bentall device 900 is configured to be re-sheathed within a modified TEVAR delivery system.

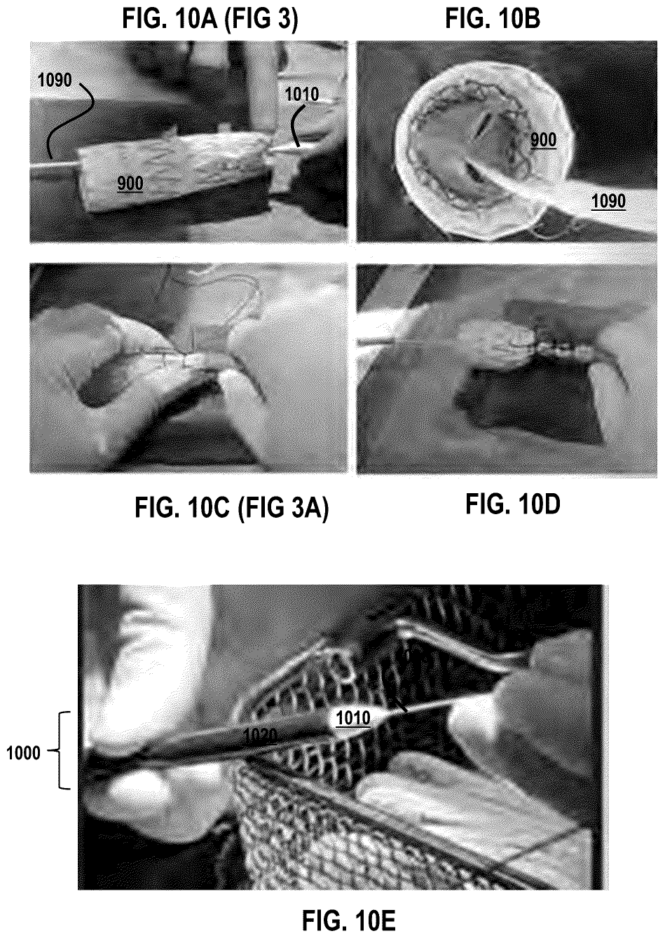

FIG. 10A through FIG. 10E are photographs that illustrate an example method for collapsing the embodiment 900 of the graft of FIG. 3 into an endovascular delivery system 1000, according to an embodiment. The delivery system 1000 includes a catheter 1090, a nosecone 1010 and a sheath 1020.

In step 511, according to the example embodiment, the delivery system 1000 is advanced via femoral artery access, and positioned in a patient's LVOT. In one example, a nosecone 1010 of the TEVAR delivery system 1000 can be trimmed to more safely advance pass the LVOT without the risk of left ventricular rupture, compared to typical devices. The graft 900 can be positioned to appropriately align the fenestrations 908 with the left and right coronary ostia. Alignment of the fenestrations can be confirmed using typical methods (e.g., using fluoroscopy) and assisted by any markers 950 present. The patient can further be overdrive-paced using a transvenous pacer and the device is deployed in the patient. If persistent endoleak is present, the coronary ostia can be accessed through the fenestrations 908 and stented with typical devices, such as covered balloon expandable stents. In at least one embodiment, no cardiopulmonary support is required.

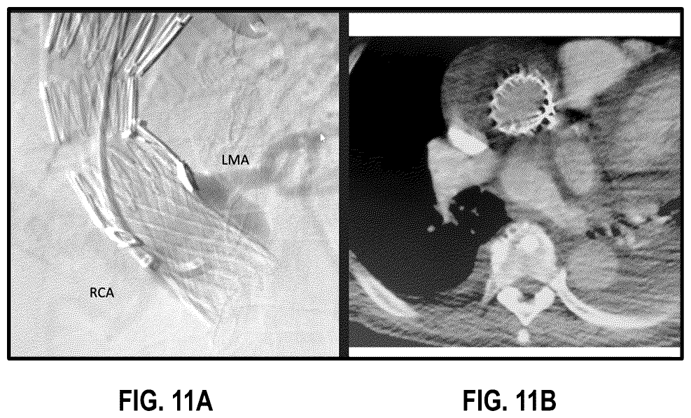

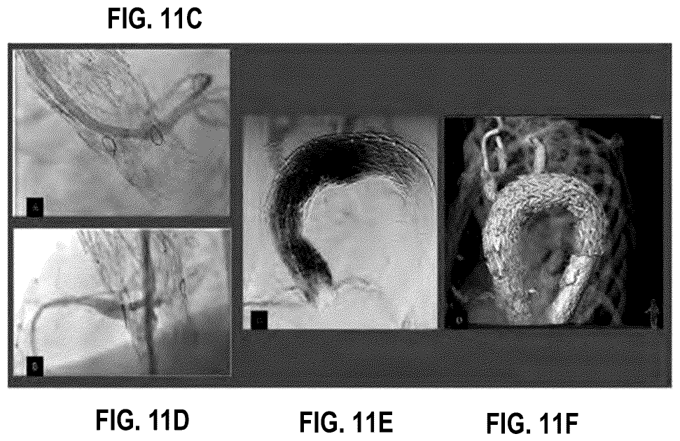

FIG. 11A through FIG. 11E are photographs that illustrate proper positioning of an embodiment 900 of the graft of FIG. 3 and adequate sealing of a false lumen after subsequent deployment of covered stents into coronary arteries, according to an embodiment.

For patient 1, the composite Endo-Bentall device 900 was advanced into the ascending aorta via the right femoral artery access. Following confirmation with fluoroscopy that the device was positioned appropriately, the patient was overdrive paced using a transvenous pacer and the device 900 was deployed. The device 900 was deployed in the aortic valve annulus and ascending aorta to the level of the innominate artery. Post-deployment echocardiography and fluoroscopy confirmed proper positioning of the composite graft as illustrated in FIG. 11A, with no evidence of aortic insufficiency, and adequate coronary perfusion through the fenestrations. The patient did not require coronary pulmonary bypass (CPB) or ECMO during the procedure. Follow-up CTA (FIG. 11B) confirmed complete thrombosis (blockage) of the false lumen, as desired, with no evidence of endoleak and with adequate coronary perfusion. Patient's chest pain resolved, and she was discharged from the hospital on post-operative day 16 without any complications. Chemotherapy and immunotherapy were resumed shortly after discharge. Patient 1 expired 5 months later due to severe urosepsis.

For patient 2, before endovascular valve replacement, an Endo-Arch graft was completed. The Endo-Arch grafting for this patient involved two procedures: 1) left common carotid artery (LCCA) to left axillary artery (LAA) bypass and 2) deployment of a thoracic branched endograft (The Gore Thoracic Branch Endoprosthesis, TBE, WL Gore, Flagstaff, AZ, USA) with the branch stent graft in the innominate artery and laser fenestration for the left subclavian artery (LSCA).

Then an Endo-Bentall device 900 was deployed. The Endo-Bentall device 900 was prepared as described above for patient 1 utilizing a 26 mm Medtronic Evolut Pro stented bioprosthetic valve as valved conduit 920 and a tapered 34-26 Cook TX2 graft that was shortened to 10 cm length as non-valved conduit 930. Two 8 mm fenestrations 908 were made in the composite device 2 cm above the base of the valve to accommodate the coronary arteries. The modified composite valved TEVAR graft 900 was re-sheathed and flushed with CO₂ and rifampin.

Ultrasound guided bilateral retrograde common femoral artery (CFA) accesses were obtained with placement of an 8 Fr sheath on the right and a 6 Fr sheath on the left. A 5 Fr sheath was placed in the left common femoral vein (CFV) for introduction of a transvenous pacemaker for rapid pacing during graft deployment. Multiple attempts were made to advance a wire from the right CFA access, however the wire repeatedly entered the false lumen of the ascending aorta. As such, a transapical approach was pursued via left anterior thoracotomy in the 5th intercostal space. The pericardium was incised and two 4-0 pledgeted Prolene sutures were placed in the apex of the heart. Systemic heparin was administered to achieve activated clotting time (ACT) greater than 250 seconds, and the apex was accessed with a 16 gauge needle. A wire was advanced into the descending thoracic aorta from the transapical access, snared and brought out through the right CFA access. The wire was then exchanged out for a Lunderquist wire. With through-and-through access established, a 34×80 mm Gore cTAG conformable thoracic stent graft was advanced into the ascending aorta from the right CFA access. The cTAG device was then deployed proximal to our previous TBE Gore graft to provide a sufficient landing zone for the Endo-Bentall graft.

The customized Endo-Bentall composite graft 900 was then advanced from the right CFA access and positioned in the landing zone of the LVOT. Positioning was confirmed with fluoroscopy ensuring the coronary fenestrations were appropriately oriented over the respective coronary ostia. The patient was overdrive paced and the Endo-Bentall graft 900 deployed. Post-deployment echocardiography and angiography confirmed proper positioning of the device, adequate coronary perfusion through the fenestrations and no aortic insufficiency.

In later stages, a second Gore cTAG device (34×100 mm) was deployed from the sinotubular junction to the innominate artery as depicted in FIG. 4F by an additional stent 450 in that first branch off the aortic arch. Finally, another 34×100 mm cTAG device was deployed to extend the distal extent of the repair to the descending aorta as depicted in FIG. 4F as extended graft 430. Completion angiography again demonstrated adequate flow through the coronaries, left subclavian artery (LSCA), left carotid artery (LCCA) and innominate artery, good aortic valve function and complete seal of the ascending and aortic arch aneurysm. Two overlapping covered stents (Viabahn 5.0×19 mm and iCAST 6.0×19 mm) into the left main coronary artery and one covered stent (iCAST 6.0×19 mm) into the right coronary artery were necessary for adequate sealing of the false lumen as depicted in FIG. 4F by the coronary artery stents 420.

The later stage stenting enabled by the device 900 is further illustrated in FIG. 11C which depicts overlapping covered stents into the left main coronary artery and FIG. 11D which shows one covered stent into the right coronary stent. FIG. 11E depicts the final angiography; and FIG. 11F depicts the final 3-D reconstructed CT scan.

Patient 2 was discharged from the hospital a week later without any complications. A 3-month follow-up CTA still demonstrated the desired complete false lumen thrombosis.

### Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### 4. REFERENCES

Each of the following references is hereby incorporated by reference as if fully recited herein, except for terminology inconsistent with that used herein.
1. Gaia D F, Bernal O, Castilho E, Ferreira C B N D, Dvir D, Simonato M, Palma J H. First-in-Human endo-Bentall procedure for simultaneous treatment of the ascending aorta and aortic valve. JACC Case Rep. 2020; 2:480-485
2. Gandet T, Westermann D, Conradi L, Panuccio G, Heidemann F, Rohlffs F, Kolbel T. Modular endo-Bentall procedure using a “rendez-Vous access”. J Endovasc Ther. 2022; 29:711-716
3. Vallee A, Guihaire J, Ghostine S, Fabre D, Haulon S. A patient-specific proof of concept with a three-dimentionally printed model before performing an endovascular Bentall procedure. J Vasc Surg Cases Innov Tech. 2021; 7:750-754.
4. Ali N, Hildick-Smith D, Parker J, Malkin C J, Cunnington M S, Gurung S, Mailey J, MacCarthy P A, Bharucha A, Brecker S J, Hoole S P, Dorman S, Doshi S N, wiper A, Buch M H, Banning A P, Spence M S, Blackman D J. Long—term durability of self-expading and balloon expandable transcatheter aortic valve prosthesis: UK TAVI registry. Catheter Cardiovasc Interv. 2023; 101:932-942.
5. Leshnower B G, Duwayri Y M, Nicholson W J, Ueyama H, Gleason P, Shekiladze N, Greenbaum A B, Babaliaros V. Endo-Bentall procedure using off-the-shelf catheter devices to repair an Aorto-Atrial fistula. Circulation: Cardio Vasc Interv. 2023; 16:891-897.
6. Stankovic G, colombo A, Presbitero P, Branden F V D, Inglese L, Cernigliaro C, Niccoli L, Bartorelli A L, rubartelli P, Reifart N, Heyndrickx G R, Saunamaki K, Morice M C, Sgura F A, Mario C D. Randomized evaluation of polytetrafluoroethylene-covered stent in saphenous vein grafts: the Randomized Evaluation of polytetrafluoroethylene-COVERed stent in Saphenous vein grafts (RECOVERS) Trial. Circulation. 2003;8:37-42.

What is claimed is:

1. An apparatus for endovascular repair of aorta root, the apparatus comprising:
a self-expanding valved conduit configured for endovascular placement within an aortic valve of a subject, said valved conduit including a valve between a distal end of the valved conduit and a proximal end of the valved conduit and including a fenestration disposed between the valve and the proximal end of the valved conduit, the fenestration configured for allowing fluid flow from inside the valved conduit to a coronary artery of the subject; and
a self-expanding non-valved conduit configured for endovascular placement within an aortic root of the subject;
wherein
the proximal end of the valved conduit is fixed inside a distal end of the non-valved conduit and the distal end of the valved conduit extends outside the distal end of the non-valved conduit, and
a fenestration is disposed in the non-valved conduit in fluid communication with the fenestration in the valved conduit.

2. The apparatus as recited in claim 1, wherein the fenestration disposed in the non-valved conduit is wire-reinforced.

3. The apparatus as recited in claim 1, wherein the distal end of the non-valved conduit is tapered smaller than a proximal end of the non-valved conduit.

4. The apparatus as recited in claim 1, wherein:
the proximal end of the valved conduit is fixed inside the distal end of the non-valved conduit by a suture disposed between the valve and the proximal end of the valved conduit, which suture attaches the valved conduit to the non-valved conduit.

5. The apparatus as recited in claim 1, wherein the apparatus is configured to be compressed inside an endovascular delivery subsystem.

6. A system comprising:
the apparatus as recited in claim 5; and
the endovascular delivery subsystem
wherein, the apparatus is compressed inside the endovascular delivery subsystem.
